(19) United States
(12) Patent Application Publication
(10) Pub. No.: US 2023/0263728 A1
(43) Pub. Date: Aug. 24, 2023

(54) BIOLOGICALLY ACTIVE ADDITIVE BASED ON MESOPOROUS SILICON DIOXIDE AND ON MICELLAR SOLUTIONS OF ACTIVE SUBSTRATES AND METHOD FOR PREPARING SAME

(71) Applicant: DGP Technology Limited, Mahe (SC)

(72) Inventor: Thomas RIVIELLO, Gonzales, TX (US)

(21) Appl. No.: 17/677,160

(22) Filed: Feb. 22, 2022

(51) Int. Cl. A61K 9/107; A61K 47/26; A61K 47/02; A61K 36/9066; A61K 36/36; A61K 31/7048; A61K 31/12

(52) U.S. Cl. CPC A61K 9/1075; A61K 47/26; A61K 47/02; A61K 36/9066; A61K 36/36; A61K 31/7048; A61K 31/12

(57) ABSTRACT

The group of inventions provides a biologically active additive and a method for preparing same. The biologically active additive is a micellar solution of an active substance in at least one emulsifier selected from the group comprising: polysorbate 20, polysorbate 80, polysorbate 20 and polysorbate 80, the emulsifier being adsorbed on amorphous mesoporous silicon dioxide nanoparticles having mesopore size from 0.2 to 1.8 nm by way of stirring in a disperser. The active substance may be selected from the group comprising: curcumin extract and rutin extract, dihydroquercetin extract, dihydroquercetin extract with quercetin extract, mixed tocopherol concentrate, cannabis oil with cannabidiol level up to 85%, cannabidiol isolate, cannabidiol isolate with mixed tocopherol concentrate, ADEK vitamin mixture concentrate, Sophora japonica extract, rosemary extract, cholecalciferol, thioctic acid, ferulic acid, resveratrol, resveratrol with ferulic acid, boswellia extract, betulin extract, icariin extract, chlorophyll extract, cannabidiol isolate with melatonin, cannabis oil having cannabidiol level up to 85% with passionflower extract, vardenafil, tadalafil, curcumin extract with soy lecithin, paclitaxel extract, collagen extract with soy lecithin. A method for preparing a biologically active additive comprises loading, into the disperser, an active substance in at least one emulsifier selected from the group comprising: polysorbate 20, polysorbate 80, polysorbate 20 and polysorbate 80, heating to a temperature of 80-90° C. while stirring continuously at from about 1000 rpm to about 2000 rpm, cooling to a temperature from below 60° C. to below 45° C., adding amorphous mesoporous silicon dioxide having a mesopore size from 0.2 to 1.8 nm, stirring at from about 1000 rpm to about 2000 rpm for about 0.5 h, followed by an increase in the stirring speed to 7000-9000 rpm until producing a homogeneous product in the form of a loose powder. The group of inventions provides improved bioavailability of the active substance.

The group of inventions relates to the field of food industry and pharmacology, animal husbandry, agriculture, and discloses the preparation of a biologically active additive used in the above fields.

The present application discloses the use of a biologically active additive in the food industry and (or) pharmacology.

Solubility and bioavailability of active pharmaceutical substances are the most significant criteria in the development of finished pharmaceutical products, since poor solubility typically leads to decreased bioavailability. Unfortunately, there is currently no universal solution to improve the solubility and, consequently, bioavailability of active substrates.

Known are various compositions used both in the food industry and in pharmaceutics comprising various active substances of multidirectional action.

For example, known is a composition of curcumin in combination with mixed tocopherols, which provides an anti-inflammatory effect (CA 2595860 A1).

Known is a composition comprising curcumin, rutin, thioctic acid, which has antioxidant, immunomodulatory, anti-inflammatory effects (U.S. Ser. No. 10/980,791 B1).

RU 2615815 C2 provides the use of emulsifiers polysorbate 80 and polysorbate 20 for producing a dosage form; the dosage form is produced by way of heating, stirring, followed by cooling and diluting with water.

All of the above compositions have insufficient bioavailability, low digestibility in the gastrointestinal tract.

The technical problem is the insufficient bioavailability of active substances that are used as biologically active additives or as pharmaceutical products. This problem also presents: ineffective use of both biologically active additives and pharmaceutical products as intended, insufficient effect on the human (or animal) organism, weakened preventive, therapeutic and other effects thereof.

Provided is a biologically active additive that is a micellar solution of an active substance in at least one emulsifier selected from the group comprising: polysorbate 20, polysorbate 80, polysorbate 20 and polysorbate 80, the emulsifier being adsorbed on amorphous mesoporous silicon dioxide nanoparticles having mesopore size from 0.2 to 1.8 nm by way of stirring in a disperser. The active substance may be selected from the group comprising: curcumin extract and rutin extract, dihydroquercetin extract, dihydroquercetin extract with quercetin extract, mixed tocopherol concentrate, cannabis oil with cannabidiol level up to 85%, cannabidiol isolate, cannabidiol isolate with mixed tocopherol concentrate, ADEK vitamin mixture concentrate, Sophora japonica extract, rosemary extract, cholecalciferol, thioctic acid, ferulic acid, resveratrol, resveratrol with ferulic acid, boswellia extract, betulin extract, icariin extract, chlorophyll extract, cannabidiol isolate with melatonin, cannabis oil having cannabidiol level up to 85% with passionflower extract, vardenafil, tadalafil, curcumin extract with soy lecithin, paclitaxel extract, collagen extract with soy lecithin.

Further provided is a method for producing a biologically active additive, the method comprising loading, into the disperser, an active substance in at least one emulsifier selected from the group comprising: polysorbate 20, polysorbate 80, polysorbate 20 and polysorbate 80, heating to a temperature of 80-90° C. while stirring continuously at from about 1,000 rpm to about 2,000 rpm, cooling to a temperature from below 60° C. to below 45° C., adding amorphous mesoporous silicon dioxide having a mesopore size from 0.2 to 1.8 nm, stirring at from about 1,000 rpm to about 2,000 rpm for about 0.5 h, followed by increasing the stirring speed to 7,000-9,000 rpm until a homogeneous product in loose powder form is produced. The active substance may be selected from the group comprising: curcumin extract and rutin extract, dihydroquercetin extract, dihydroquercetin extract with quercetin extract, mixed tocopherol concentrate, cannabis oil with cannabidiol level up to 85%, cannabidiol isolate, cannabidiol isolate with mixed tocopherol concentrate, ADEK vitamin mixture concentrate, Sophora japonica extract, rosemary extract, cholecalciferol, thioctic acid, ferulic acid, resveratrol, resveratrol with ferulic acid, boswellia extract, betulin extract, icariin extract, chlorophyll extract, cannabidiol isolate with melatonin, cannabis oil having cannabidiol level up to 85% with passionflower extract, vardenafil, tadalafil, curcumin extract with soy lecithin, paclitaxel extract, collagen extract with soy lecithin.

The technical result is a qualitative improvement in bioavailability thereby providing a significant effect of the use of (multidirectional) active substances.

Provided is a general co-grinding method (mechanochemical method). This processing method comprises: using compression energy, grating to transfer the active agent present in the micellar solution into amorphous carrier dispersed phase, and comprises a mixing step and grinding step using a ball mill, a planetary mill, or a high-intensity disperser. Thus, the carrier adsorbs micellar solutions of active substances in polysorbates into free-flowing powder form. Such a powder may be conveniently used for producing solid dosage forms while preserving the micellar structure of active agent in aqueous solution. This technology is advantageous over known methods in that it provides: complete exclusion of organic solvents from the process, two-stageness, environmental safety, scalability, flexibility of technology. The study shows the applicability of the present method for a wide range of organic compounds such as flavonoids, terpenoids, curcuminoids, glycosides, aglycones, vitamins, polyphenolic resins, natural heterocyclic and carbocyclic acids, which bear a huge pharmacological potential. Finally, we studied the comparative bioavailability of: curcumin powder, micellar curcumin solution in polysorbates, and nanopowder of sorbent deposited by the above method with micellar curcumin solution in tweens on male humans aged 27 to 43 years, in groups, each consisting of nine subjects. The products were taken orally by weighed portion of 1 g. It should be noted that the organic phase of micellar curcumin on mesoporous silicon dioxide is completely soluble in water to form a stable suspension.

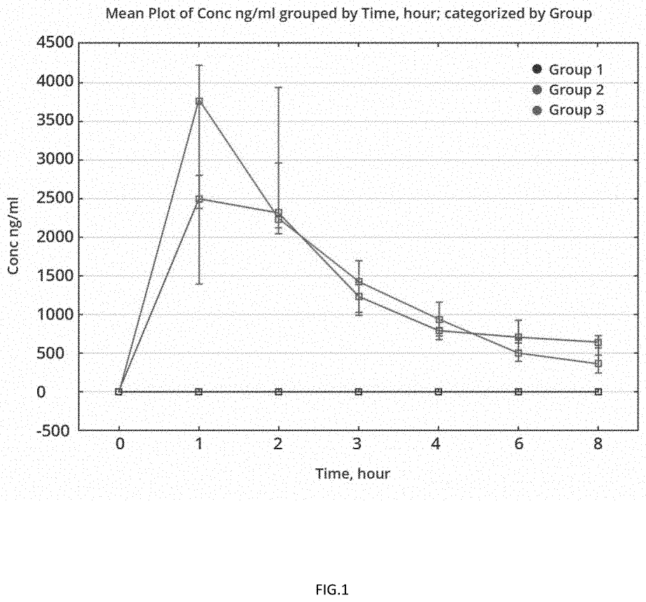

The graph (FIG. 1) shows data on bioavailability of various forms of curcumin over time.

Green graph: the group that was administered with a micellar curcumin solution in polysorbates.

Red graph: the group that was administered with a micellar curcumin solution on mesoporous silicon dioxide.

Blue graph: the group that was administered with curcumin extract dry powder.

As follows from the diagram, there is a correlation of data of the green graph and red graph, which fact thus makes it possible to conclude that the behaviors of the provided forms of curcumin in the gastrointestinal tract are repeatable, and makes it possible to state that the method of synthesis of solid dispersions based on mesoporous silicon dioxide and micellar solutions of active substrates in polysorbates is applicable and versatile. The latter acts as a stabilizer of the amorphous and colloidal states in the form of nanoscale micelles.

Other active substances according to the subject group of inventions, that are fixed onto a stabilizer, i.e. mesoporous silicon dioxide, show similar bioavailability data.

### EXPERIMENTAL

1. A weighed portion of curcumin extract in the amount of 1.1 g (11%) and rutin extract in the amount was 0.3 g (3%) was dissolved while continuously stirring and heating in polysorbate 80 at 1,000 rpm to a temperature of 80° C. The resulting dark solution (10 g) was cooled to a temperature below 60° C. and added to 15 g of amorphous silicon dioxide having mesopore size from 0.2 to 1.8 nm while stirring in the IKA T 50 digital disperser for 0.5 h at 1,000 rpm. The speed was then increased to 8,000 rpm for increased uniformity of substances distribution. At 10 g (40%):15 g (60%) ratio, a yellow loose product was produced.

2. A weighed portion of dihydroquercetin extract in the amount of 1 g (10%) was dissolved while continuously stirring and heating in polysorbate 20 at 2,000 rpm to a temperature of 90° C. The resulting dark solution (10 g) was cooled to a temperature below 45° C. and added to 20 g of amorphous silicon dioxide having mesopore size from 0.2 to 1.8 nm while stirring in the IKA T 50 digital disperser for 0.5 h at 2,000 rpm. The speed was then increased to 8,000 rpm for increased dispersibility and uniformity of loose powder distribution.

3. A weighed portion of dihydroquercetin extract in the amount of 0.5 g (5%) and quercetin extract (0.5 g (5%) was dissolved while continuously stirring at 1,500 rpm and heating to 85° C. in a mixture of polysorbate 80 and polysorbate 20. The resulting solution (10 g} was cooled and added to 15 g of mesoporous silicon dioxide while continuously stirring in the IKA T 50 digital disperser at 1,500 rpm for 0.5 h. The stirring speed was then increased to 8,000 rpm; at 10 g (40%):15 g (60%) ratio, a yellow loose powder was produced.

4. A weighed portion of mixed tocopherol concentrate in the amount of 1.5 g (15%) was dissolved while continuously stirring and heating to 90° C. in a mixture of polysorbates 80 and 20, at 1,500 rpm. The resulting solution (10 g) was cooled to a temperature below 50° C. and added to 15 g of amorphous silicon dioxide having mesopore size from 0.2 to 1.8 nm while stirring in the IKA T 50 digital disperser for 0.5 h at 2,000 rpm. The speed was then increased to 8,000 rpm for improved dispersibility of reactants. At 10 g (40%):15 g (60%) ratio, a yellow loose powder was produced.

5. A weighed portion of cannabis oil having cannabidiol level up to 85% in the amount of 2 g (20%) was dissolved in polysorbate 80 while continuously stirring at 1,000 rpm and heating to 80° C. The resulting solution in the amount of 10 g was cooled to a temperature below 55° C. and added to 15 g of amorphous silicon dioxide having mesopore size from 0.2 to 1.8 nm while stirring in the IKA T 50 digital disperser for 0.5 h at 1,500 rpm. The speed was then increased to 8,000 rpm for increased uniformity of reactant distribution. At 10 g (40%):15 g (60%) ratio, a dark-yellow loose powder was produced.

6. A weighed portion of cannabidiol isolate in the amount of 2 g (20%) was dissolved in polysorbate 20 while continuously stirring and heating. The resulting slightly colored solution (10 g) was cooled to a temperature below 45° C. and added to 15 g of mesoporous silicon dioxide while stirring for 0.5 h in the IKA T 50 digital disperser. The speed was then increased to 8,000 rpm for increased dispersibility of substances. At 10 g (40%):15 g (60%) ratio, a light loose powder was produced.

7. A weighed portion of cannabidiol isolate in the amount of 1.8 g (18%) and 0.2 g (2%) of mixed tocopherol concentrate was dissolved in a mixture of polysorbates 80 and 20 while stirring at 2,000 rpm and heating to 80° C. The resulting dark solution in the amount of 10 g was cooled to a temperature below 60° C. and added to 15 g of amorphous silicon dioxide having mesopore size from 0.2 to 1.8 nm while stirring at 2,000 rpm for 0.5 h in the IKA T 50 digital disperser. The stirring speed was then increased to 9,000 rpm to reduce clustering. At 10 g (40%):15 g (60%) ratio, a yellow loose powder was produced.

8. A weighed portion of ADEK vitamin mix concentrate in the amount of 2 g (20%) was dissolved in polysorbate 80 while continuously stirring at 1,500 rpm and heating to 85° C. The resulting solution (10 g) was cooled to a temperature below 45° C. and added to 15 g of mesoporous silicon dioxide while stirring in the IKA T 50 digital disperser for 0.5 h at 1,000 rpm. The speed was then increased to 9,000 rpm for improved dispersibility of the substances. At 10 g (40%):15 g (60%) ratio, a yellow loose powder was produced.

9. A weighed portion of Sophora japonica extract in the amount of 1 g (10%) was dissolved while continuously stirring at 2,000 rpm and heating to 90° C. in polysorbate 20. The resulting dark solution (10 g) was cooled to a temperature below 60° C. and added to 20 g of amorphous silicon dioxide having mesopore size from 0.2 to 1.8 nm while stirring in the IKA T 50 digital disperser for 0.5 h at 1,000 rpm. The speed was then increased to 8,000 rpm for increased dispersibility and uniformity of distribution of substances. At 10 g (33.3%):20 g (66.6%) ratio, a light yellow loose powder was produced.

10. A weighed portion of rosemary extract in the amount of 1 g (10%) was dissolved while continuously stirring at 1,500 rpm and heating to 80° C. in a mixture of polysorbate 80 and polysorbate 20. The resulting solution (10 g) was cooled to a temperature below 55° C. and added to 15 g of amorphous silicon dioxide having mesopore size from 0.2 to 1.8 nm while stirring in the IKA T 50 digital disperser for 0.5 h at 1,000 rpm. The speed was then increased to 8,000 rpm for improved dispersibility of reactants. At 10 g (40%):15 g (60%) ratio, a yellow loose powder was produced.

11. A weighed portion of cholecalciferol (vitamin D3) in the amount of 1.5 g (15%) was dissolved while stirring at 2,000 rpm and heating to 90° C. in a mixture of polysorbate 80 and polysorbate 20. The resulting solution (10 g) was cooled to a temperature below 50° C. and added to 20 g of amorphous silicon dioxide having mesopore size from 0.2 to 1.8 nm while stirring vigorously in the IKA T 50 digital disperser for 0.5 h at 1,500 rpm. The speed was then increased to 9,000 rpm to improve dispersibility of reactants. At 10 g (33.3%):20 g (66.6%) ratio, a white loose powder was produced.

12. A weighed portion of thioctic acid in the amount of 1.5 g (15%) was dissolved while continuously stirring at 1,000 rpm and heating to 80° C. in a mixture of polysorbate 80 and polysorbate 20. The resulting solution (10 g) was cooled to a temperature below 50° C. and added to 15 g of amorphous silicon dioxide having mesopore size from 0.2 to 1.8 nm while stirring in the IKA T 50 digital disperser for 0.5 h at 1,500 rpm. The speed was then increased to 8,000 rpm to improve dispersibility of reactants. At 10 g (40%):15 g (60%) ratio, a while loose powder was produced.

13. A weighed portion of ferulic acid in the amount of 1.5 g (15%) was dissolved while continuously stirring at 1,000 rpm and heating to 85° C. in a mixture of polysorbate 80 and polysorbate 20. The resulting solution (10 g) was cooled to a temperature below 45° C. and added to 15 g of amorphous silicon dioxide having mesopore size from 0.2 to 1.8 nm while stirring in the IKA T 50 digital disperser for 0.5 h at 1,500 rpm. The speed was then increased to 8,000 rpm for improved dispersibility of reactants. At 10 g (40%):15 g (60%) ratio, a while loose powder was produced.

14. A weighed portion of resveratrol in the amount of 1.5 g (15%) was dissolved while continuously stirring at 1,000 rpm and heating to 85° C. in a mixture of polysorbate 80 and polysorbate 20. The resulting solution (10 g) was cooled to a temperature below 45° C. and added to 15 g of amorphous silicon dioxide having mesopore size from 0.2 to 1.8 nm while stirring in the IKA T 50 digital disperser for 0.5 h at 1,000 rpm. The speed was then increased to 8,000 rpm for improved dispersibility of reactants. At 10 g (40%):15 g (60%) ratio, a while loose powder was produced.

15. A weighed portion of resveratrol in the amount of 0.5 g (5%) and ferulic acid in the amount of 0.5 g (5%) was dissolved while stirring at 1,500 rpm and heating to 90° C. in a mixture of polysorbate 80 and polysorbate 20. The resulting solution (10 g) was cooled to a temperature below 50° C. and added to 20 g of amorphous silicon dioxide having mesopore size from 0.2 to 1.8 nm while stirring vigorously in the IKA T 50 digital disperser for 0.5 h at 2,000 rpm. The speed was then increased to 9,000 rpm to improve dispersibility of reactants. At 10 g (33.3%):20 g (66.6%) ratio, a white loose powder was produced.

16. A weighed portion of boswellia extract in the amount of 1.2 g (12%) was dissolved while continuously stirring at 1,000 rpm and heating to 90° C. in polysorbate 80. The resulting solution (10 g) was cooled to a temperature below 50° C. and added to 15 g of amorphous silicon dioxide having mesopore size from 0.2 to 1.8 nm while stirring in the IKA T 50 digital disperser for 0.5 h at 1,000 rpm. The speed was then increased to 8,000 rpm for increased uniformity of substance distribution. At 10 g (40%):15 g (60%) ratio, a yellow loose product was produced.

17. A weighed portion of betulin extract in the amount of 1.5 g (15%) was dissolved while continuously stirring at 1,500 rpm and heating to 85° C. in a mixture of polysorbate 80 and polysorbate 20. The resulting solution (10 g) was cooled to a temperature below 45° C. and added to 15 g of amorphous silicon dioxide having mesopore size from 0.2 to 1.8 nm while stirring in the IKA T 50 digital disperser for 0.5 h at 1,500 rpm. The speed was then increased to 8,000 rpm for improved dispersibility of reactants. At 10 g (40%):15 g (60%) ratio, a while loose powder was produced.

18. A weighed portion of icariin extract in the amount of 1.5 g (15%) was dissolved while continuously stirring at 1,000 rpm and heating to 85° C. in a mixture of polysorbate 80 and polysorbate 20. The resulting solution (10 g) was cooled to a temperature below 45° C. and added to 15 g of amorphous silicon dioxide having mesopore size from 0.2 to 1.8 nm while stirring in the IKA T 50 digital disperser for 0.5 h (at 1,000 rpm). The speed was then increased to 8,000 rpm for improved dispersibility of reactants. At 10 g (40%):15 g (60%) ratio, a yellow loose powder was produced.

19. A weighed portion of chlorophyll extract in the amount of 0.5 g (5%) was dissolved while continuously stirring at 2,000 rpm and heating to 90° C. in a mixture of polysorbate 80 and polysorbate 20. The resulting dark solution (10 g) was cooled to a temperature below 50° C. and added to 15 g of amorphous silicon dioxide while continuously stirring at 2,000 rpm in the IKA T 50 digital disperser for 0.5 h. The stirring speed was then increased to 8,000 rpm. At 10 g (40%):15 g (60%) ratio, a green loose powder was produced.

20. A weighed portion of cannabidiol isolate in the amount of 1.5 g (15%) and 0.5 g (5%) of melatonin was dissolved in a mixture of polysorbate 80 and polysorbate 20 while stirring at 1,000 rpm and heating to 90° C. The resulting dark solution in the amount of 10 g was cooled to a temperature below 50° C. and added to 15 g of amorphous silicon dioxide having mesopore size from 0.2 to 1.8 nm while stirring at 1,000 rpm for 0.5 h in the IKA T 50 digital disperser. The stirring speed was then increased to 9,000 rpm to reduce clustering. At 10 g (40%):15 g (60%) ratio, a while loose powder was produced.

21. A weighed portion of cannabis oil having cannabidiol level up to 85% in the amount of 1.5 g (15%) and passionflower extract in the amount of 0.5 g (5%) was dissolved in polysorbate 80 while continuously stirring at 1,000 rpm and heating to 90° C. The resulting solution (10 g) was cooled to a temperature below 60° C. and added to 15 g of amorphous silicon dioxide having mesopore size from 0.2 to 1.8 nm while stirring at 1,000 rpm in the IKA T 50 digital disperser for 0.5 h. The speed was then increased to 8,000 rpm for increased uniformity of reactant distribution. At 10 g (40%):15 g (60%) ratio, a dark-yellow loose powder was produced.

22. A weighed portion of vardenafil in the amount of 0.5 g (5%) was dissolved in polysorbate 20 while continuously stirring at 2,000 rpm and heating to 90° C. The resulting solution (10 g) was cooled to a temperature below 60° C. and added to 15 g of amorphous silicon dioxide having mesopore size from 0.8 to 1.2 nm while continuously stirring at 1,000 rpm in the IKA T 50 digital disperser for 0.5 h. The stirring speed was increased to 7,000 rpm for complete homogenization of reaction mass. At 10 g (40%):15 g (60%) ratio, a loose powder was produced.

23. A weighed portion of tadalafil in the amount of 0.5 g (5%) was dissolved in polysorbate 20 while continuously stirring at 1,500 rpm and heating to 90° C. The resulting solution (10 g) was cooled to a temperature below 60° C. and added to 15 g of amorphous silicon dioxide having mesopore size from 0.8 to 1.2 nm while continuously stirring at 1,500 rpm in the IKA T 50 digital disperser for 0.5 h. The stirring speed was increased to 7,000 rpm for complete homogenization of reaction mass. At 10 g (40%):15 g (60%) ratio, a loose powder was produced.

24. A weighed portion of curcumin extract (1 g (10%)) and soy lecithin (0.5 g (5%)) was dissolved while heating to 80° C. and continuously stirring at 1,500 rpm in polyethylene glycol. The resulting dark solution in the amount of 10 g was cooled to a temperature below 50° C. and added to 20 g of amorphous silicon dioxide having mesopore size from 0.8 to 1.2 nm while stirring in the IKA T 50 digital disperser at 1,500 rpm for 0.5 h. The speed was then increased to 9,000 rpm to reduce clustering. At the given ratio of 10 g (33.3%) to 20 g (66.6%), a yellow loose powder was produced.

25. A weighed portion of paclitaxel extract (1 g (10%)) and soy lecithin (0.5 g (5%)) was dissolved while heating to 85° C. and continuously stirring at 1,000 rpm in polyethylene glycol. The resulting yellow solution in the amount of 10 g was cooled to a temperature below 50° C. and added to 20 g of amorphous silicon dioxide having mesopore size from 0.8 to 1.2 nm while stirring at 2,000 rpm in the IKA T 50 digital disperser for 0.5 h. The speed was then increased to 9,000 rpm to reduce clustering. At the given ratio of 10 g (33.3%) to 20 g (66.6%), a loose powder was produced.

26. A weighed portion of collagen extract (1 g (10%)) and soy lecithin (0.5 g (5%)) was dissolved while heating to 90° C. and continuously stirring at 1,000 rpm in polyethylene glycol. The resulting yellow solution in the amount of 10 g was cooled to a temperature below 45° C. and added to 20 g of amorphous silicon dioxide having mesopore size from 0.8 to 1.2 nm while stirring at 1,000 rpm in the IKA T 50 digital disperser for 0.5 h. The speed was then increased to 9,000 rpm to reduce clustering. At the given ratio of 10 g (33.3%) to 20 g (66.6%), a loose powder was produced.

### CONCLUSIONS

1. It has been experimentally shown that mechanochemical activation on a high-intensity disperser was of a general nature for a wide range of organic natural and synthetic compounds.

2. The subject technology is advantageous over known methods in that it provides: complete exclusion of organic solvents from the process, two-stageness, environmental safety, scalability, flexibility of technology.

3. The subject technology eliminates: undesirable properties of a pharmaceutical substance, undesirable side effects, unpleasant organoleptic properties.

4. Provided are increased stability under storing the active substrate and increased resistance to environmental factors (oxidative degradation).

5. The resulting powders acquire the optimal flowability required for modern high-speed tablet presses, as well as for use in hard gelatin capsules or in powders.

6. The organic phase of synthesized powders is completely soluble in water to form stable micellar solutions, the bioavailability of which correlates with the stock solutions of active substances in surfactants.

1. A biologically active additive being a micellar solution of an active substance in at least one emulsifier selected from the group comprising: polysorbate 20, polysorbate 80, polysorbate 20 and polysorbate 80, the emulsifier being adsorbed on amorphous mesoporous silicon dioxide nanoparticles having mesopore size from 0.2 to 1.8 nm by way of stirring in a disperser.

2. The biologically active additive of claim 1, wherein the active substance is selected from the group comprising: curcumin extract and rutin extract, dihydroquercetin extract, dihydroquercetin extract with quercetin extract, mixed tocopherol concentrate, cannabis oil with cannabidiol level up to 85%, cannabidiol isolate, cannabidiol isolate with mixed tocopherol concentrate, ADEK vitamin mixture concentrate, Sophora japonica extract, rosemary extract, cholecalciferol, thioctic acid, ferulic acid, resveratrol, resveratrol with ferulic acid, boswellia extract, betulin extract, icariin extract, chlorophyll extract, cannabidiol isolate with melatonin, cannabis oil having cannabidiol level up to 85% with passionflower extract, vardenafil, tadalafil, curcumin extract with soy lecithin, paclitaxel extract, collagen extract with soy lecithin.

3. A method for producing a biologically active additive of claim 1, the method comprising loading, into the disperser, an active substance in at least one emulsifier selected from the group comprising: polysorbate 20, polysorbate 80, polysorbate 20 and polysorbate 80, heating to a temperature of 80-90° C. while stirring continuously at from about 1,000 rpm to about 2,000 rpm, cooling to a temperature from below 60° C. to below 45° C., adding amorphous mesoporous silicon dioxide having a mesopore size from 0.2 to 1.8 nm, stirring at from about 1,000 rpm to about 2,000 rpm for about 0.5 h, followed by increasing the stirring speed to 7,000-9,000 rpm until a homogeneous product in loose powder form is produced.

4. A method for preparing the biologically active additive of claim 3, wherein the active substance is selected from the group comprising: curcumin extract and rutin extract, dihydroquercetin extract, dihydroquercetin extract with quercetin extract, mixed tocopherol concentrate, cannabis oil with cannabidiol level up to 85%, cannabidiol isolate, cannabidiol isolate with mixed tocopherol concentrate, ADEK vitamin mixture concentrate, Sophora japonica extract, rosemary extract, cholecalciferol, thioctic acid, ferulic acid, resveratrol, resveratrol with ferulic acid, boswellia extract, betulin extract, icariin extract, chlorophyll extract, cannabidiol isolate with melatonin, cannabis oil having cannabidiol level up to 85% with passionflower extract, vardenafil, tadalafil, curcumin extract with soy lecithin, paclitaxel extract, collagen extract with soy lecithin.
